(19) United States
(12) Patent Application Publication
(10) Pub. No.: US 2025/0332443 A1
(43) Pub. Date: Oct. 30, 2025

(54) METHODS FOR MICROBIAL CONTROL AND RESIN CURING

(71) Applicants: George Edward Parris, Gaithersburg, MD (US); Sina Mossahebi, Towson, MD (US)

(72) Inventors: George Edward Parris, Gaithersburg, MD (US); Sina Mossahebi, Towson, MD (US)

(21) Appl. No.: 19/187,999

(22) Filed: Apr. 24, 2025

Related U.S. Application Data
(60) Provisional application No. 63/638,381, filed on Apr. 24, 2024.

(51) Int. Cl. A61N 5/10; A61B 17/88; A61L 27/36; A61L 27/54; A61L 27/58

(52) U.S. Cl. CPC A61N 5/10; A61B 17/88; A61L 27/365; A61L 27/54; A61L 27/58; A61L 2430/02; A61N 2005/1098

(57) ABSTRACT

Methods for controlling microbial organisms and curing polymeric resins via targeted proton beam irradiation are disclosed. In one aspect, microbial control is achieved by irradiating a target region with a proton beam configured to deliver a precise Bragg Peak dose, effectively eliminating pathogens while minimizing damage to surrounding tissues. In another aspect, the invention details methods for curing liquid acrylic bone cement during orthopedic procedures including kyphoplasty, vertebroplasty, and screw fixation. The proton beam initiates polymerization without chemical catalysts, controlling heat generation and enhancing precision. The invention further provides for activating bioactive therapeutic precursor molecules and visualizing energy deposition using leuco-crystal violet dye. These integrated approaches enhance therapeutic outcomes across multiple medical applications.

### FIELD

The present invention relates to methods for controlling microbial organisms and methods for curing resins. More specifically, the present invention relates to the use of proton beam irradiation for the aforementioned methods, particularly in medical and related applications.

### BACKGROUND

Proton beam therapy is a recognized cancer treatment modality. Proton beams, which are positively charged particles, exhibit a characteristic depth-dose profile. This depth-dose profile is defined by the Bragg Peak, a phenomenon where the majority of the proton beam's energy is deposited within a localized volume at a specific depth in tissue. This property of localized energy deposition offers advantages in cancer therapy by enabling targeted irradiation of tumors while reducing radiation exposure to surrounding healthy tissues. Clinical applications of proton beam therapy are primarily focused on the treatment of various malignant tumors.

Outside of cancer therapy, the potential of proton beams has not been extensively investigated. Conventional approaches to treating microbial infections rely heavily on antibiotic agents. However, the increasing prevalence of antibiotic-resistant microorganisms poses a significant challenge to effective infection management. Antibiotic resistance reduces the efficacy of standard antibiotic therapies, leading to prolonged infections, increased morbidity, and higher healthcare costs. Furthermore, effective treatment of localized infections can be limited by factors such as inadequate drug penetration into certain tissues or the presence of physical barriers surrounding the infection site. Localized infections, including osteomyelitis, abscesses, and tuberculomas, often present challenges for conventional antibiotic delivery and achieving therapeutic drug concentrations at the site of infection.

In separate medical and industrial fields, polymeric resins are utilized in various applications, including orthopedic procedures. Acrylic resins, for example, are commonly used as bone cements in osteoplasty and joint replacement surgeries. Current methods for curing these resins typically involve the use of chemical catalysts. These chemical catalysts are mixed with the liquid resin immediately prior to application to initiate polymerization and solidification. However, the use of chemical catalysts presents certain limitations. Chemical curing processes can be difficult to control precisely, often proceeding rapidly after catalyst addition. The exothermic nature of resin polymerization can lead to substantial heat generation, which may be detrimental to surrounding tissues. Furthermore, the presence of residual chemical catalyst within the cured resin may raise biocompatibility concerns in medical applications. Existing radiofrequency activation methods for resin curing also exhibit limitations similar to chemical catalysts, particularly in terms of controlling the curing process and potential heat generation.

Therefore, a need exists for novel methods that can address the challenges associated with controlling microbial organisms, particularly in localized infections and against antibiotic-resistant strains. A further need exists for improved methods for curing polymeric resins, especially in medical procedures such as osteoplasty, that offer enhanced control over the curing process, reduce heat generation, and minimize or eliminate the need for chemical catalysts.

### SUMMARY

The present disclosure envisages a method for controlling microbial organisms comprises irradiating a target region containing microbial organisms with a proton beam from an external source, wherein the proton beam is configured and directed to deliver a Bragg Peak dose of radiation selectively to the target region, and wherein the Bragg Peak dose is effective to control the microbial organisms.

In one embodiment, the method further comprises inhibiting the growth of the microbial organisms.

In one embodiment, the method comprises eliminating the microbial organisms.

In one embodiment, the method is for treating a localized infection in vivo in a patient, wherein the target region is an infected tissue region.

In one embodiment, the infected tissue region is selected from the group consisting of bone, cyst, abscess, tuberculoma, organ, cavity, and sinus.

In one embodiment, the microbial organisms are selected from the group consisting of bacteria, fungi, viruses, and parasites.

In one embodiment, the microbial organisms are antibiotic-resistant microorganisms.

In one embodiment, the Bragg Peak dose of radiation is in a range from 1 Gray to 60 Gray, therapeutically effective for inhibiting growth or killing the microorganisms in vivo while minimizing damage to healthy tissue surrounding the infected tissue region.

In one embodiment, the localized infection is osteomyelitis, and the infected tissue region is bone tissue.

In one embodiment, the localized infection is selected from the group consisting of tuberculoma, abscess, cyst, sinus infection, gangrene, and intracellular viral infection.

In one embodiment, the method is for inhibiting microbial growth in vitro, wherein the target region is a sample containing microbial organisms within a sealed container.

In one embodiment, the sealed container is a medical device package.

In one embodiment, the sample comprises a pharmaceutical composition.

In one embodiment, the method further comprises activating a chemical reaction in vivo wherein a bioactive therapeutic precursor molecule is converted to an active therapeutic agent by irradiation with the proton beam.

In one embodiment, the bioactive therapeutic precursor molecule is converted to an antibiotic agent upon irradiation with the proton beam.

The present disclosure further envisages a method for performing osteoplasty in vivo in a patient comprises preparing a bone defect site in the patient requiring osteoplasty, introducing a liquid acrylic bone cement into the bone defect site, and curing the liquid acrylic bone cement in situ within the bone defect site by irradiating the bone defect site with a controlled proton beam from an external source, wherein the controlled proton beam is configured and directed to deliver a Bragg Peak dose to the acrylic bone cement to selectively initiate polymerization and solidification of the acrylic bone cement.

In one embodiment, the acrylic bone cement is cured without requiring a separate chemical catalyst mixed with the acrylic bone cement prior to its introduction into the bone defect site.

In one embodiment, the cured acrylic bone cement provides structural support and fixation to promote bone healing at the bone defect site.

In one embodiment, the osteoplasty is for replacement of a joint selected from the group consisting of hip joint, knee joint, shoulder joint, and elbow joint.

In one embodiment, the method further comprises incorporating leuco-crystal violet into the liquid acrylic bone cement, wherein the leuco-crystal violet provides visualization of the acrylic bone cement or the proton beam path during the osteoplasty procedure. Care should be taken to avoid contacting the leuco-crystal violet with blood.

In one embodiment, the osteoplasty procedure is selected from the group consisting of kyphoplasty and vertebroplasty.

In one embodiment, the method further comprises containing the liquid acrylic bone cement within an expandable plastic bladder prior to introduction into the bone defect site, wherein the plastic bladder prevents the liquid acrylic bone cement from spreading beyond an intended area.

In one embodiment, the bone defect site includes a pre-drilled hole in the bone, and wherein the method further comprises placing a screw in the bone and introducing the liquid acrylic bone cement to enhance bonding of the screw to the bone.

In one embodiment, the method further comprises placing a plurality of screws in one or more bones, aligning the plurality of screws, and curing the liquid acrylic bone cement to secure the plurality of screws in their aligned positions.

In one embodiment, the method is for prophylactically strengthening normal bones.

The present disclosure further envisages a method for visualizing energy deposition from a proton beam in biological tissue during in vivo therapeutic treatment comprises incorporating leuco-crystal violet dye into a biocompatible material placed within or adjacent to the biological tissue, irradiating the biological tissue and the biocompatible material with a proton beam, and observing a color change in the leuco-crystal violet dye in the biocompatible material, the color change being indicative of energy deposition from the proton beam within the biological tissue and the biocompatible material.

In one embodiment, the biocompatible material is an acrylic resin.

### BRIEF DESCRIPTION OF THE DRAWINGS

FIG. 1 is a schematic diagram illustrating the experimental setup for agar tube experiments designed to evaluate the effects of proton beams on microbial growth in relation to the Bragg Peak, according to an embodiment of the present invention.

FIG. 2 is a diagrammatic comparison of expected microbial growth and photographs of actual agar tubes, demonstrating the Bragg Peak effect, according to an embodiment of the present invention.

FIG. 3 is a photograph of three agar tubes from an experiment demonstrating microbial growth inhibition, according to an embodiment of the present invention.

FIG. 4 is a comparative photograph of two agar tubes, labeled “S1” and “SC control”, highlighting the Bragg Peak effect, according to an embodiment of the present invention.

FIG. 5 is a photograph of an agar tube labeled “S2”, providing further visual evidence of the Bragg Peak effect, according to an embodiment of the present invention.

FIG. 6 comprises three photographic images depicting the experimental setup for Petri dish experiments designed to assess the antimicrobial effect at varying depths, according to an embodiment of the present invention.

FIG. 7 is a photograph of a Petri dish labeled “1” after irradiation and incubation, demonstrating widespread bacterial growth at a shallow depth, according to an embodiment of the present invention.

FIG. 8 is a photograph of a Petri dish labeled “3” after irradiation and incubation, showing reduced bacterial growth and an asymmetric pattern at an intermediate depth, according to an embodiment of the present invention.

FIG. 9 is a photograph of a Petri dish labeled “8” after irradiation and incubation, demonstrating significant microbial growth inhibition near the Bragg Peak depth, according to an embodiment of the present invention.

FIG. 10 comprises photographs of four Petri dishes containing bone slices, comparing irradiated samples with non-irradiated control samples and demonstrating reduced microbial growth in irradiated samples.

FIG. 11 comprises photographs of actual bone slices comparing irradiated samples with non-irradiated control samples and visually confirming reduced microbial contamination on irradiated bone slices.

FIG. 12 illustrates a flow diagram depicting method for performing osteoplasty in vivo in a patient, according to an embodiment of the present invention.

FIG. 13 illustrates a flow diagram depicting method for visualizing energy deposition from a proton beam in biological tissue during in vivo therapeutic treatment, according to an embodiment of the present invention.

### DETAILED DESCRIPTION

It has been discovered that proton beams, when configured to deliver a Bragg Peak dose of radiation to a target region, can effectively control microbial organisms and cure resins. This discovery reveals a utility of proton beams beyond cancer therapy. Prior to this discovery, the potential of proton beams for controlling microbial organisms and inducing resin curing was not fully recognized or exploited. This discovery arises from an understanding of the interaction of proton beams with matter and the resulting chemical and biological effects.

The chemical effect of proton beams involves the release of free electrons as protons disrupt electrons bound to molecules in the medium through which the proton passes. These free electrons act as free radicals and cause a variety of chemical reactions among the molecules of the medium. It has been demonstrated that proton beams can catalyze polymerization of acrylic polymers, even at moderate and sub-ambient temperatures. This capability, coupled with the ability of proton beams to deliver high density of energy and free electrons deep within media of density approximately 1 g/mL, provides a tool for localized chemical reactions and biological effects. This capability for inducing chemical reactions extends beyond polymerization and can include the activation of bioactive precursor molecules, potentially enabling targeted therapeutic effects through selective irradiation of specific chemical compounds in vivo.

The interaction of proton beams with matter is characterized by a physical phenomenon. As positively charged protons traverse a medium, they interact with the atoms and molecules of that medium through electromagnetic forces. This interaction leads to the disruption of electrons bound within atoms and molecules, resulting in the ejection of these electrons. These ejected electrons, now unbound and possessing energy, are termed free electrons. These free electrons are chemically reactive species, behaving as free radicals. Due to their unpaired electron configuration, they participate in a variety of chemical reactions, including oxidation, reduction, and polymerization. The specific chemical reactions induced depend on the composition of the medium being irradiated and the energy spectrum of the proton beam. Experiments have shown that proton beams can catalyze the polymerization of acrylic polymers. This polymerization catalysis by proton beams occurs even at moderate temperatures, such as room temperature (approximately 25 degrees Celsius), and even at sub-ambient temperatures (below 25 degrees Celsius). This capability is noted as conventional polymerization processes often require chemical initiators and/or elevated temperatures to proceed at a practical rate. The capacity of proton beams to induce polymerization at moderate and low temperatures highlights their potential for applications where temperature-sensitive materials or biological tissues are involved. Proton beams possess the characteristic of depth-dose deposition, described by the Bragg Peak. This property allows for the delivery of high density of energy within a localized volume at a controllable depth within media having a density similar to biological tissue (approximately 1 g/mL). This localized energy deposition results in a localized generation of free electrons, enabling spatially controlled chemical reactions and biological effects within the target region. This combination of chemical reactivity and localized energy delivery makes proton beams a tool.

A factor contributing to the absence of proton beam application in infection therapy relates to the traditional separation of disciplines among physicists generating the beams, oncologists treating cancer, and infectious disease specialists. The present invention bridges this gap through detailed analysis of the biological mechanisms underlying cellular response to radiation. Analysis of biochemical pathways indicates that radiation-induced damage to cellular organelles, specifically mitochondria and lysosomes, results in elevated concentrations of reactive oxygen species (ROS). These ROS subsequently interact with cellular DNA, potentially causing structural damage (chromothripsis), activation of apoptotic pathways, or cellular dysfunction. Cellular DNA exhibits heightened vulnerability to ROS during the replication phase of the cell cycle. Consequently, radiation demonstrates enhanced efficacy against rapidly dividing cells, including numerous tumor types, microbial organisms, virus-infected cells, and certain multicellular parasites that undergo rapid proliferation.

Experimentation with proton beams directed against bacteria and fungi demonstrates potential medical utility against various infections in humans, animals, and plants. The efficacy of such applications is particularly pronounced in localized infections and in conditions where antimicrobial agents exhibit limited effectiveness or where restricted blood flow impedes conventional treatment modalities.

In addition to direct microbial control, proton beams can activate chemical reactions in vivo wherein bioactive therapeutic precursor molecules are converted to active therapeutic agents. The same mechanism of free electron generation that enables microbial control and resin polymerization can induce chemical transformations of specific precursor molecules. For example, certain antibiotic precursor molecules, designed to be inert during administration, can be selectively activated at infection sites through targeted proton beam irradiation. This approach enables localized antibiotic activity while minimizing systemic effects and potential toxicity associated with conventional antibiotic administration.

The total applied proton dosage for effective inhibition of microorganisms in the affected region may be up to 60 Gray (Gy), with the specific dosage depending on the type of infective agent being targeted. For clinical applications, these dosages are typically administered in fractionated treatments, with daily doses as low as 1 Gy. This fractionated approach allows for cumulative antimicrobial effects while minimizing potential radiation effects on surrounding healthy tissues. The specific dosage regimen may be tailored based on factors including the microbial species involved, the extent of infection, the anatomical location, and patient-specific considerations such as age and general health status.

In one aspect, the present invention provides a method for controlling microbial organisms by irradiating a target region containing the organisms with a proton beam. The method includes irradiating a target region containing microbial organisms with a proton beam from an external source. The proton beam is configured and directed to deliver a Bragg Peak dose of radiation selectively to the target region. The Bragg Peak dose is effective to control the microbial organisms. This method is applicable for treating localized infections in vivo in a patient, or for inhibiting microbial growth in vitro in a sample, such as a sample contained within a sealed container.

In one aspect, the present invention discloses a method for controlling microbial organisms. This method is designed for antimicrobial applications. The method is for controlling the presence and proliferation of microbial organisms, including bacteria, fungi, viruses, and parasites. The method includes the step of irradiating a defined “target region” that contains or is suspected to contain microbial organisms. The irradiation uses a “proton beam,” generated and directed from an “external source.” The proton beam is “configured and directed” to achieve delivery of a “Bragg Peak dose of radiation.” The Bragg Peak dose is delivered “selectively to the target region.” This targeting uses the Bragg Peak phenomenon to concentrate the radiation dose, and antimicrobial effect, within the intended target volume. The “Bragg Peak dose,” when delivered in this targeted manner, is “effective to control the microbial organisms.” The term “effective to control” encompasses levels of microbial control, including inhibiting microbial growth and eliminating (killing) the organisms. The method is applicable across diverse scenarios, including:
Treating localized infections in vivo in a patient: Therapeutic applications for treating infections within living organisms, including humans and animals, for localized infections such as osteomyelitis, abscesses, and tuberculomas.
Inhibiting microbial growth in vitro in a sample: Non-therapeutic applications outside of living organisms, focused on controlling microbial populations in samples including research samples, industrial samples, or materials requiring sterilization.

Inhibiting microbial growth in vitro in a sample, such as a sample contained within a sealed container: Specific in vitro applications where the sample requiring microbial control is enclosed within a “sealed container,” relevant for sterilization applications, such as sterilizing medical devices within packaging, or sterilizing pharmaceutical compositions in vials or ampoules. The use of a sealed container ensures maintenance of sterility post-irradiation.

The method of the present invention demonstrates particular applicability to the following pathological conditions:

Tuberculomas: Tuberculomas comprise nodular structures typically forming in pulmonary or cerebral tissues wherein the host immune system creates a barrier around infection foci caused by Mycobacterium tuberculosis. Such infections frequently demonstrate antimicrobial resistance and reduced vascular perfusion, thereby limiting the effectiveness of conventional antibiotic therapy. Proton beams can access these localized infection sites while maintaining precise dose delivery control.

Cysts, abscesses and sinuses: Infectious processes may establish within anatomical sinuses where immune system access is compromised. Formation of cystic structures surrounding infection foci (exemplified by Staphylococcus aureus with antimicrobial resistance) results in abscess development. Conventional treatment often necessitates surgical drainage procedures that present risk of infection dissemination. Proton beam application to these localized infections provides a non-invasive or minimally invasive alternative, potentially reducing infection spread risk and associated complications.

Intracellular viral and bacterial infections: Viral pathogens invade host cells and modulate cellular apoptotic mechanisms, thereby facilitating viral replication while evading host immune surveillance. The accelerated DNA synthesis characteristic of virally infected cells increases cellular susceptibility to radiation-induced damage. Analogous mechanisms occur in parasitic infections such as malaria, wherein Plasmodium sporozoites invade hepatocytes and utilize host cellular machinery for replication and production of exoerythrocytic merozoites. The elevated DNA synthesis in these cellular environments potentially enhances susceptibility to proton beam radiation effects.

Gangrene: Gangrenous conditions represent infection complications associated with traumatic injury wherein vascular compromise prevents adequate tissue perfusion. Resultant tissue hypoxia creates conditions favorable for anaerobic microbial proliferation while simultaneously limiting immune system access. Proton beam delivery functions independently of vascular systems, thereby providing a treatment modality unaffected by local perfusion status.

Penetrating trauma: Traumatic injuries caused by projectiles, explosions, or similar mechanisms frequently introduce environmental contaminants resistant to complete surgical debridement. Similar contamination risks exist in compound fractures and traumatic disruption of integumentary or visceral structures. Proton beam treatment directed at traumatized tissues and identifiable foreign materials may provide prophylactic antimicrobial effects, potentially reducing infection incidence in tissues with compromised vascular supply, such as partially amputated extremities.

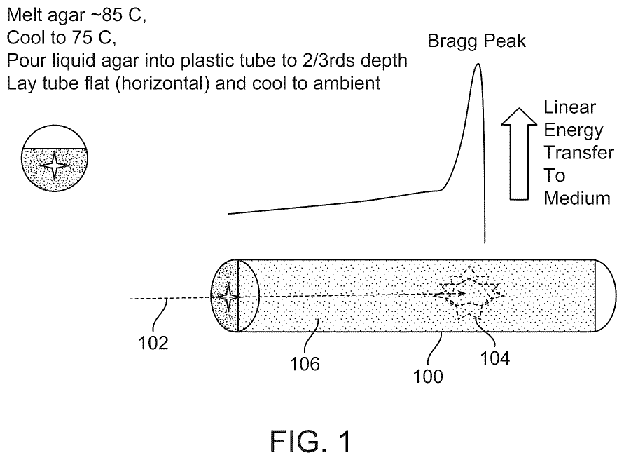

FIG. 1 is a schematic diagram illustrating the experimental setup for agar tube experiments. The diagram depicts an agar tube (100) oriented horizontally. A proton beam (102) is shown directed transversely to the agar tube (100). The anticipated Bragg Peak (104) is diagrammatically represented by a star-like shape within the agar medium (106) inside the agar tube (100). An arrow indicates the linear energy transfer to the medium. This schematic diagram depicts the experimental model used to investigate the Bragg Peak effect on microbial organisms. The horizontal orientation of the agar tube (100) and the transverse direction of the proton beam (102) were designed to position the Bragg Peak (104) within the agar medium (106), thereby allowing for localized irradiation and subsequent assessment of microbial growth patterns in relation to the anticipated Bragg Peak location.

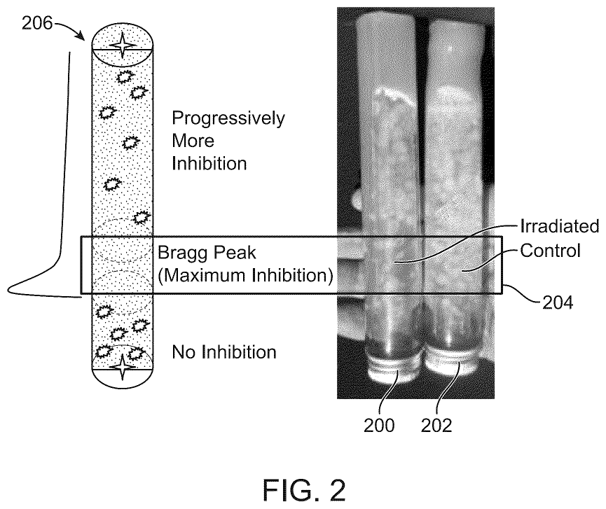

FIG. 2 is a diagrammatic comparison of expected microbial growth and photographs of actual agar tubes. The diagrammatic portion (206) illustrates the expected pattern of microbial growth inhibition in relation to the Bragg Peak. The diagrammatic portion (206) depicts progressively more inhibition approaching the Bragg Peak, maximum inhibition at the Bragg Peak region (204), and no inhibition beyond the Bragg Peak. The photographic portion of FIG. 2 juxtaposes an image of an irradiated agar tube (200) and an image of a control agar tube (202) for providing visual confirmation of the Bragg Peak effect. The irradiated agar tube (200) visually demonstrates reduced microbial colony density in the Bragg Peak region (204), corresponding to the zone of maximum inhibition as predicted diagrammatically in the diagrammatic portion (206). In contrast, the control agar tube (202) exhibits a uniform microbial colony density throughout the agar medium. FIG. 2 provides visual evidence that proton beam irradiation, when configured to deliver a Bragg Peak dose, can effectively control microbial organisms, specifically by inhibiting microbial growth in the region of maximum energy deposition.

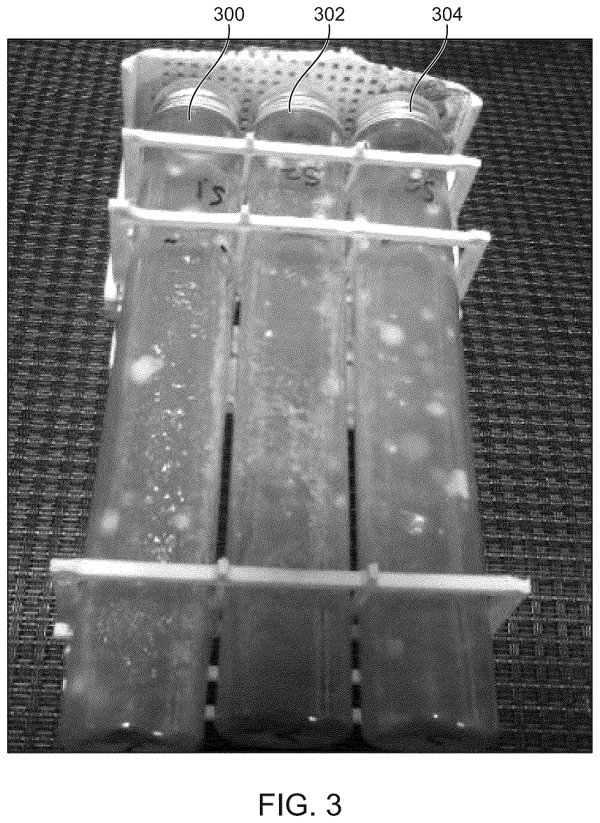

FIG. 3 shows a photograph of three agar tubes (300, 302, 304) from an experiment similar to that shown in FIG. 2, further demonstrating the microbial growth inhibition effect. The photograph compares two irradiated samples (300, 302), labeled “S” on the tubes, with a control sample (304), labeled “C” on the tube. The agar tubes (300, 302, 304) are shown mounted in a white rack for observation. The irradiated agar tubes (300, 302) visually exhibit a reduced density of microbial colonies, particularly in the central region of the tubes corresponding to the expected Bragg Peak location, as compared to the control agar tube (304), which shows a more uniform and denser distribution of microbial colonies. FIG. 3 provides further visual corroboration of the microbial growth inhibition effect observed in the agar tube model, supporting the broad applicability of the proton beam antimicrobial method.

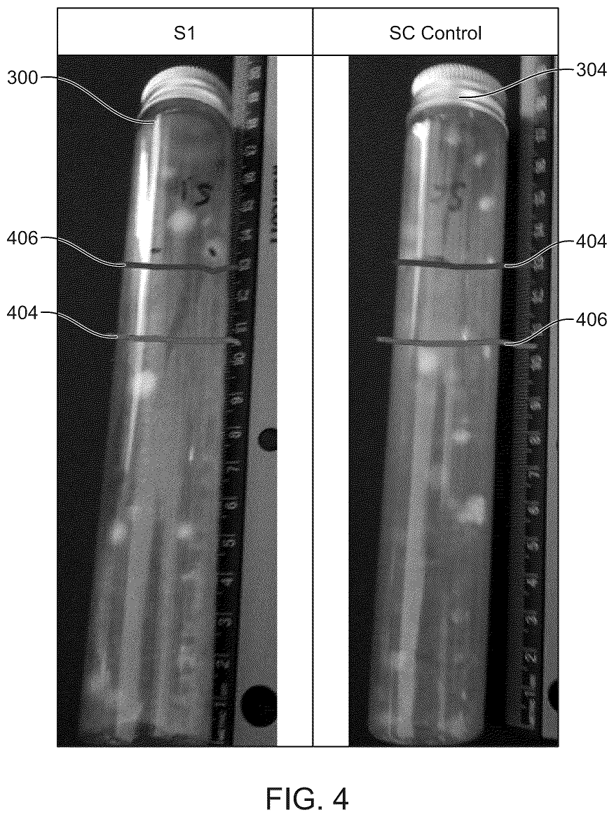

FIG. 4 presents a comparative photograph of two agar tubes, specifically labeled “S1” (300) to indicate a first irradiated sample and “SC control” (304) to indicate a control sample, from an experiment designed to further highlight the Bragg Peak effect. Lines (404) are superimposed on both tubes S1 and SC (300, 304) to indicate the expected location of the Bragg Peak, and lines (406) are superimposed to indicate a point beyond the Bragg Peak region. The irradiated agar tube “S1” (300) shows a visually discernible reduction in microbial colony density in the region around the line (404), corresponding to the expected Bragg Peak location. In contrast, the control agar tube “SC control” (304) exhibits a more uniform colony density across the region corresponding to both the line (404) and line (406), indicating a lack of localized microbial growth inhibition in the absence of proton beam irradiation.

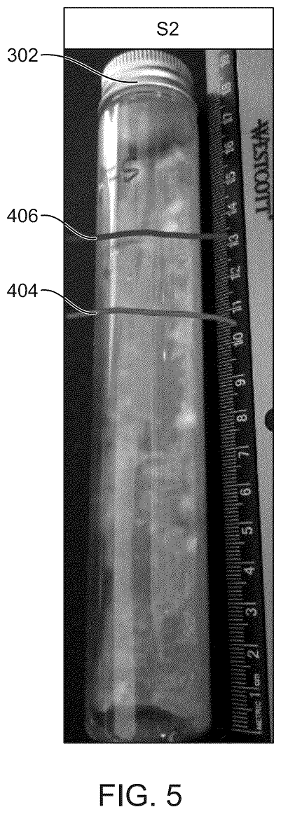

FIG. 5 shows a photograph of an agar tube labeled “S2” (302), representing a second irradiated sample from the same experiment as the “S1” sample shown in FIG. 4. Similar to the “S1” sample (300 in FIG. 4), the agar tube “S2” (302) also exhibits a visually apparent reduction in microbial colony density in the region around the superimposed line (404, indicating Bragg Peak location), further corroborating the localized microbial growth inhibition effect induced by proton beam irradiation at the Bragg Peak.

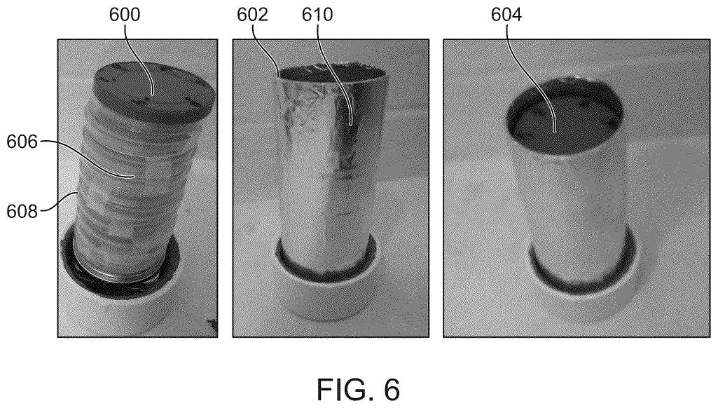

FIG. 6 illustrates the experimental setup for Petri dish experiments designed to assess the antimicrobial effect of proton beams at varying depths. FIG. 6 comprises three photographic images (600, 602, 604) depicting a stack of Petri dishes (606) inoculated with fecal bacteria. The Petri dishes (606) are shown contained within an opaque plastic tube (608). Image (602) shows the stack of Petri dishes (606) and opaque plastic tube (608) further wrapped in aluminum foil (610), representing a shielded condition. Image (604) shows the stack of Petri dishes (606) and opaque plastic tube in an upright orientation. The experimental setup, as depicted in FIG. 6, was designed to simulate different tissue depths by varying the position of Petri dishes within the stack and to investigate the effect of shielding using aluminum foil. This experimental design allowed for the assessment of proton beam antimicrobial efficacy at varying depths and under different conditions, providing data relevant to in vivo applications where tissue depth and shielding effects are important considerations.

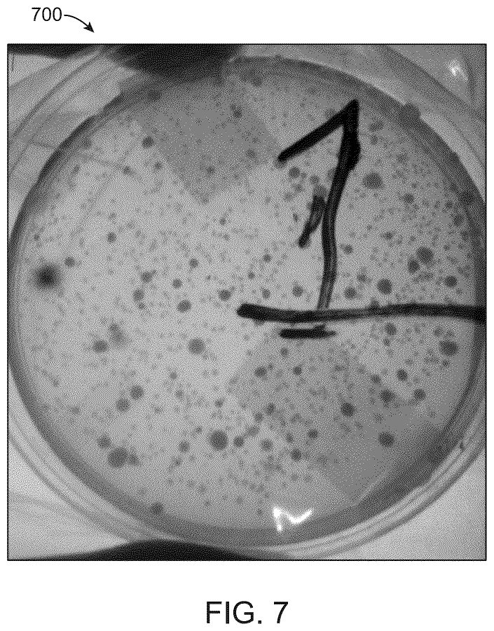

FIG. 7 shows a photograph of a Petri dish labeled “1” (700) after irradiation and incubation. The Petri dish (700) exhibits widespread bacterial growth across the agar surface. Petri dish “1” (700) represents the topmost dish in the stack, corresponding to a shallow depth of penetration for the proton beam. The photograph of Petri dish “1” (700) depicts widespread bacterial growth across the agar surface, indicating minimal antimicrobial effect at this shallow depth.

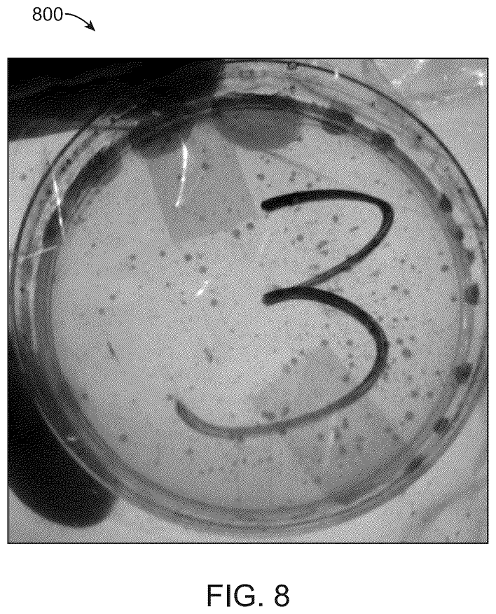

FIG. 8 shows a photograph of a Petri dish labeled “3” (800) after irradiation and incubation. The Petri dish (800) exhibits reduced bacterial growth and an asymmetric pattern of bacterial colonies across the agar surface. Petri dish “3” (800) represents a dish located at an intermediate depth within the stack. The photograph of Petri dish “3” (800) exhibits reduced bacterial growth as compared to Petri dish “1” (700 in FIG. 7), indicating a partial antimicrobial effect at this intermediate depth. Furthermore, the bacterial growth in Petri dish “3” (800) shows an asymmetric pattern, suggesting variations in radiation dose around the perimeter of the dish due to the irradiation setup.

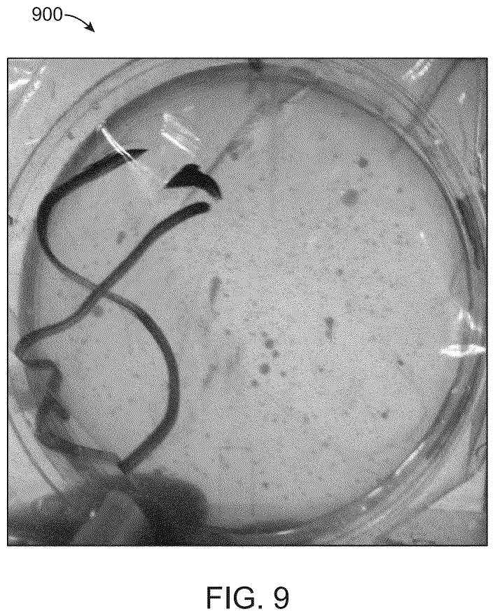

FIG. 9 shows a photograph of a Petri dish labeled “8” (900) after irradiation and incubation. The Petri dish (900) exhibits significantly reduced bacterial colonies on the agar surface, with only a few isolated colonies visible. Petri dish “8” (900) represents a dish positioned further down in the stack, approximating the expected Bragg Peak depth for the proton beam energy used in the experiment. The photograph of Petri dish “8” (900) demonstrates significantly reduced bacterial colonies on the agar surface, with only a few isolated colonies visible. FIG. 9 provides strong visual evidence of substantial microbial growth inhibition in Petri dish “8” (900), directly supporting the effectiveness of proton beam irradiation at the Bragg Peak in controlling microbial organisms, and demonstrating the potential for achieving a high degree of microbial control through targeted Bragg Peak irradiation.

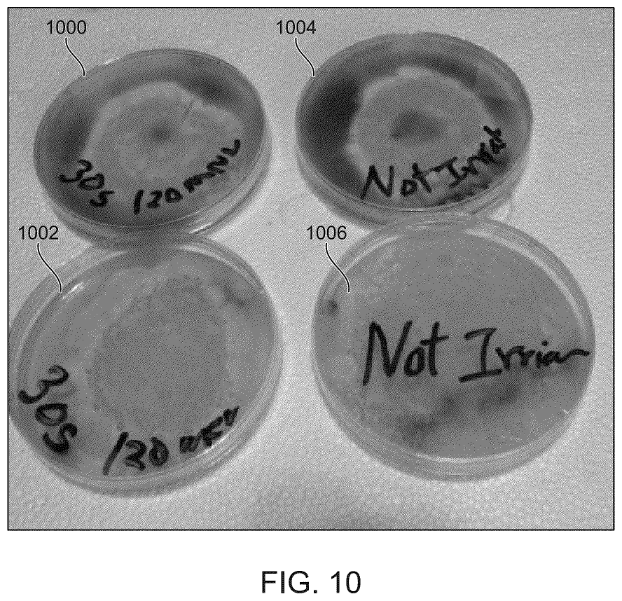

FIG. 10 presents photographs of four Petri dishes (1000, 1002, 1004, 1006) containing bone slices exposed to soil microbes, comparing irradiated and non-irradiated samples. The Petri dishes labeled “30S 120 MeV” (1000, 1002) contain irradiated bone slice samples, indicating samples irradiated for 30 seconds with a 120 MeV proton beam. The Petri dishes labeled “Not Irrad” (1004, 1006) represent non-irradiated control samples. The photographs in FIG. 10 visually demonstrate less microbial growth (1008) in the Petri dishes containing irradiated bone slices (1000, 1002) as compared to the denser microbial growth (1010) observed in the Petri dishes containing non-irradiated control bone slices (1004, 1006). FIG. 10 provides visual evidence supporting the effectiveness of proton beam irradiation in controlling microbial organisms within bone tissue, a clinically relevant tissue type for localized infections.

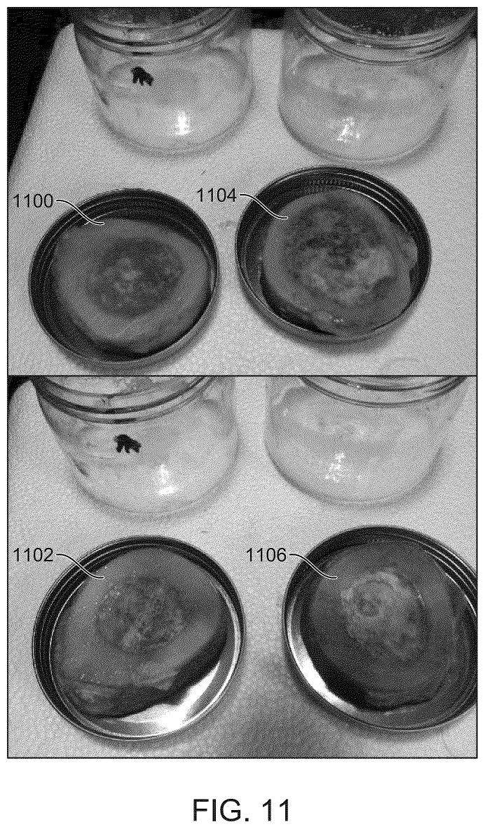

FIG. 11 shows photographs of the actual bone slices (1100, 1102, 1104, 1106) described in FIG. 10, providing a closer view of the bone slice samples themselves. The bone slices on the left (1100, 1102) represent irradiated samples, corresponding to the Petri dishes labeled “30S 120 MeV” in FIG. 10. The bone slices on the right (1104, 1106) represent non-irradiated control samples, corresponding to the Petri dishes labeled “Not Irrad” in FIG. 10. The photographic images in FIG. 11 visually confirm reduced microbial contamination on the irradiated bone slices (1100, 1102) as compared to the non-irradiated bone slices (1104, 1106). FIG. 11 provides further detailed visual corroboration of the reduced microbial burden on irradiated bone tissue, strengthening the experimental support for in vivo applications of the proton beam antimicrobial method, particularly for localized infections in bone tissue such as osteomyelitis. The experimental data presented in FIGS. 10 and 11, in conjunction with the agar tube and Petri dish experiments, collectively demonstrate the broad applicability and effectiveness of the proton beam antimicrobial method.

The method of the present invention also includes applications beyond medical treatment of human and animal infections. One such example includes botanical and structural wood infestation. More specifically, ligneous structures, including living trees and manufactured wood products, are subject to infestation by various organisms including termites and fungi. While chemical pesticides provide control options in certain circumstances, proton beam application offers a methodology for infestation eradication without additional structural damage to the wood substrate. In colonial insects such as termites, where reproduction is centralized in a single reproductive female, targeted proton beam application to the brood chamber can effectively eliminate the entire colony through interruption of reproductive capacity.

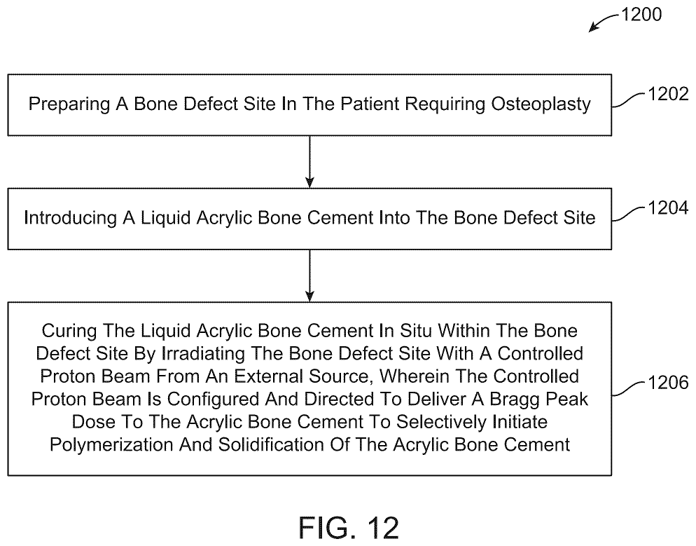

In another aspect, the invention provides a method for performing osteoplasty in vivo in a patient. FIG. 12 illustrates a flow diagram depicting method (1200) for performing osteoplasty in vivo in a patient. At block (1202), the method (1200) comprises preparing a bone defect site in the patient requiring osteoplasty. At block (1204), the method (1200) includes introducing a liquid acrylic bone cement into the bone defect site. At block (1206), the method (1200) curing the liquid acrylic bone cement in situ within the bone defect site by irradiating the bone defect site with a controlled proton beam from an external source. In one embodiment, the controlled proton beam is configured and directed to deliver a Bragg Peak dose to the acrylic bone cement to selectively initiate polymerization and solidification of the acrylic bone cement.

In specific embodiments, the method for performing osteoplasty is applied to kyphoplasty and vertebroplasty procedures. These procedures involve the repair of compressed or fractured vertebrae, typically resulting from osteoporosis, trauma, or tumor involvement. The controlled nature of proton beam activation provides particular advantages in these procedures, where precise cement placement and controlled curing are critical to successful outcomes.

In another embodiment, the liquid acrylic bone cement is contained within an expandable plastic bladder prior to introduction into the bone defect site. The bladder serves to prevent the liquid cement from spreading beyond the intended area, addressing a significant challenge in conventional bone cement applications where unintended cement migration can lead to complications such as nerve compression or vascular compromise. Following placement of the bladder containing the liquid cement, the proton beam is directed to cure the cement in situ, providing controlled expansion and solidification within the confined space of the bladder.

The method is further applicable to enhancing the fixation of orthopedic hardware, particularly screws used in bone fixation. In this embodiment, a pre-drilled hole in the bone receives a screw, and liquid acrylic bone cement is introduced to enhance the bonding interface between the screw and surrounding bone tissue. This application is particularly valuable in cases of osteoporotic bone or revision surgeries where screw purchase may be compromised. The method allows for placement of multiple screws in one or more bones, their precise alignment, and subsequent secure fixation through controlled proton beam curing of the surrounding cement.

The method also encompasses prophylactic bone strengthening applications, wherein normal bones subject to increased mechanical stress or at risk of fracture due to underlying conditions such as osteopenia or metastatic disease are reinforced through the selective application of proton beam-cured acrylic cement. This preventative approach may reduce fracture risk in vulnerable patient populations without the need for more invasive surgical interventions.

The method (1200) cures the acrylic bone cement without requiring a separate chemical catalyst mixed with the acrylic bone cement prior to introduction into the bone defect site. The cured acrylic bone cement provides structural support and fixation to promote bone healing at the bone defect site. The osteoplasty is for replacement of a joint selected from the group consisting of hip joint, knee joint, shoulder joint, and elbow joint. The osteoplasty may specifically involve kyphoplasty or vertebroplasty for vertebral repair, or may be applied to enhance screw fixation in bone or prophylactically strengthen normal bones at risk of fracture.

While the primary objective of the method for performing osteoplasty, as depicted in FIG. 12, is to provide structural support and fixation for bone repair or joint replacement, it is recognized that surgical procedures involving bone and tissue manipulation inherently carry a risk of microbial contamination and subsequent infection. Therefore, the use of proton beam irradiation in the osteoplasty method, as described herein, offers a potential secondary benefit in controlling microbial organisms at the surgical site.

As demonstrated in the experimental examples and supported by the evidence in FIGS. 2-11, proton beam irradiation, when configured to deliver a Bragg Peak dose, is effective in inhibiting growth or eliminating microbial organisms. By utilizing proton beam irradiation to cure the acrylic bone cement in the osteoplasty method, a localized dose of radiation is delivered to the bone defect site and surrounding tissues. This radiation dose, while primarily intended to initiate resin polymerization and solidification, may also exert an antimicrobial effect, potentially reducing the risk of post-operative infection at the surgical site. This potential antimicrobial co-benefit of the proton beam irradiation in osteoplasty is particularly relevant considering the challenges associated with surgical site infections, especially in orthopedic procedures involving implants and bone grafts.

TABLE 1 below indicates appropriate/effective dosages (Gy) for treating infections and administration in fractionated dosages (Gy/day).

TABLE 1
APPROPRIATE DOSAGES AND CLINICAL PRACTICES

| | | Total Dose | |
|---|---|---|---|
| Infection | Location | Gy | Clinical Practice |
| Bacteria | Soft tissue | 60 | Fractionated to 1 Gy/day as appropriate |
| Bacteria | Bone or Cartilage | 70 | Fractionated to 1-1.5 Gy/day depending on |
| | | | tolerance |
| Virus | Soft tissue | 40 | 1.8-2 Gy/day; consider hypo if urgent |
| | | | eradication |
| Virus | Bone or Cartilage | 50 | 1.8-2 Gy/day |
| Fungus | Soft tissue | 60 | 1 Gy/day or 2 Gy every other day to avoid |
| | | | tissue toxicity |
| Fungus | Bone or Cartilage | 70 | 1 Gy/day; hyperfractionation may help limit |
| | | | necrosis |
| Intracellular | Soft tissue | 45 | 1.5-2 Gy/day |
| Parasite | | | |
| Intracellular | Bone or Cartilage | 55 | 1.5-2 Gy/day |
| Parasite | | | |
| Multicellular | Soft tissue | 50 | 2 Gy/day or hypo-fractionated to 5 Gy × 10 |
| Parasite (worm, | | | |
| insect) | | | |
| Multicellular | Bone or Cartilage | 60 | 2 Gy/day or hypo-fractionated; consider |
| Parasite (worm, | | | tissue sensitivity |
| insect) | | | |
| Biological | Ex vivo container | 60 | Single fraction of 60 Gy (sterilization, no |
| Sample | | | tissue concern) |
| Infestation | Living Plant or | 60 | Single fraction of 60 Gy (used in |
| (fungus, termite) | Wood | | agriculture/materials) |

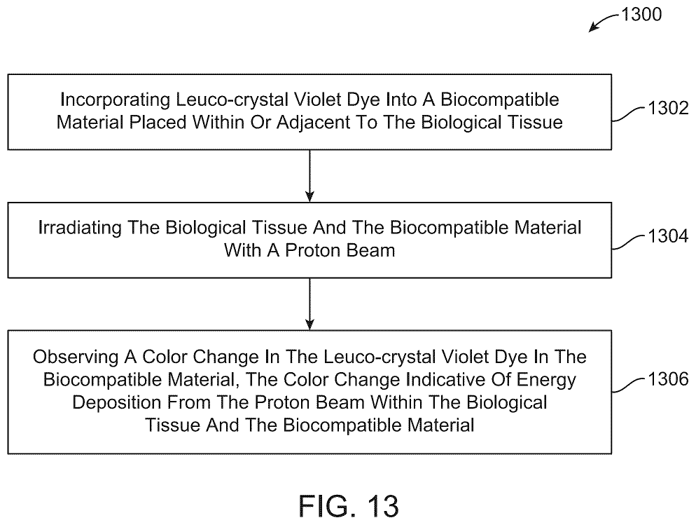

In a further aspect, the invention provides a method (1300) for visualizing energy deposition from a proton beam in biological tissue during in vivo therapeutic treatment. FIG. 13 illustrates a flow diagram depicting method (1300) for visualizing energy deposition from a proton beam in biological tissue during in vivo therapeutic treatment. At block (1302), the method (1300) comprises incorporating leuco-crystal violet dye into a biocompatible material placed within or adjacent to the biological tissue. At block (1304), the method (1300) comprises irradiating the biological tissue and the biocompatible material with a proton beam. At block (1306), the method (1300) comprises observing a color change in the leuco-crystal violet dye in the biocompatible material, the color change indicative of energy deposition from the proton beam within the biological tissue and the biocompatible material. The biocompatible material can be an acrylic resin.

In the context of the method for controlling microbial organisms, the visualization method (1300) can be employed to ensure accurate and targeted delivery of the proton beam to the infected tissue region in vivo. By incorporating leuco-crystal violet dye into a biocompatible material placed within or adjacent to the infected tissue region, the surgeon or clinician can visually monitor the energy deposition pattern of the proton beam. Observing the color change in the leuco-crystal violet dye allows for confirmation that the Bragg Peak dose is being delivered precisely to the intended target region containing microbial organisms. This visual feedback can be used to optimize treatment planning and execution, ensuring that the radiation dose is concentrated at the infection site to maximize microbial control while minimizing exposure of surrounding healthy tissues to radiation. The visualization method, therefore, directly contributes to improving the therapeutic efficacy and safety of the proton beam antimicrobial method in vivo.

Similarly, in the context of the method (1200) for performing osteoplasty, the visualization method (1300) provides a valuable tool for enhancing the precision and control of resin curing. By incorporating leuco-crystal violet dye into the liquid acrylic bone cement prior to introduction into the bone defect site, the surgeon can visually monitor the location and distribution of the acrylic bone cement in vivo during the osteoplasty procedure. Upon irradiation with the proton beam to initiate curing, the color change in the leuco-crystal violet dye provides real-time feedback on the energy deposition pattern within the acrylic bone cement and surrounding bone tissue. This visual information allows the surgeon to verify that the proton beam is correctly positioned and that the Bragg Peak dose is being delivered to the acrylic bone cement to achieve complete and localized curing in situ. Visualization of energy deposition, therefore, assists in ensuring accurate and controlled curing of the acrylic bone cement in osteoplasty procedures, contributing to the structural integrity and therapeutic outcome of the bone repair or joint replacement.

What is claimed is:

1. A method for controlling microbial organisms, the method comprising:
irradiating a target region containing microbial organisms with a proton beam from an external source, wherein the proton beam is configured and directed to deliver a Bragg Peak dose of radiation selectively to the target region, and wherein the Bragg Peak dose is effective to control the microbial organisms.

2. The method of claim 1, wherein the step of controlling microbial organisms comprises inhibiting growth of the microbial organisms.

3. The method of claim 1, wherein the step of controlling microbial organisms comprises eliminating the microbial organisms.

4. The method of claim 1, wherein the method is for treating a localized infection in vivo in a patient, and wherein the target region is an infected tissue region.

5. The method of claim 4, wherein the infected tissue region is selected from the group consisting of bone, cyst, abscess, tuberculoma, organ, cavity, and sinus.

6. The method of claim 4, wherein the microbial organisms are selected from the group consisting of bacteria, fungi, viruses, and parasites.

7. The method of claim 6, wherein the microbial organisms are antibiotic-resistant microorganisms.

8. The method of claim 4, wherein the Bragg Peak dose of radiation is in a total dose range from 45 Gray to 70 Gray, therapeutically effective for inhibiting growth or killing of the microorganisms in vivo while minimizing damage to healthy tissue surrounding the infected tissue region, and
wherein the dose is delivered in fractions 1 Gray to 2 Gray per day.

9. The method of claim 4, wherein the localized infection is osteomyelitis, and the infected tissue region is bone tissue.

10. The method of claim 4, wherein the localized infection is selected from the group consisting of tuberculoma, abscess, cyst, sinus infection, gangrene, and intracellular viral infection.

11. The method of claim 1, wherein the method is for inhibiting microbial growth in vitro, and wherein the target region is a sample containing microbial organisms within a sealed container.

12. The method of claim 11, wherein the sealed container is a medical device package.

13. The method of claim 11, wherein the sample comprises a pharmaceutical composition.

14. The method of claim 1, further comprising activating a chemical reaction in vivo wherein a bioactive therapeutic precursor molecule is converted to an active therapeutic agent by irradiation with the proton beam.

15. The method of claim 14, wherein the bioactive therapeutic precursor molecule is converted to an antibiotic agent upon irradiation with the proton beam.

16. A method for performing osteoplasty in vivo in a patient, the method comprising:
preparing a bone defect site in the patient requiring osteoplasty;
introducing a liquid acrylic bone cement into the bone defect site; and
curing the liquid acrylic bone cement in situ within the bone defect site by irradiating the bone defect site with a controlled proton beam from an external source, wherein the controlled proton beam is configured and directed to deliver a Bragg Peak dose to the acrylic bone cement to selectively initiate polymerization and solidification of the acrylic bone cement.

17. The method of claim 16, wherein the acrylic bone cement is cured without requiring a separate chemical catalyst mixed with the acrylic bone cement prior to introduction into the bone defect site.

18. The method of claim 16, wherein the cured acrylic bone cement provides structural support and fixation to promote bone healing at the bone defect site.

19. The method of claim 16, wherein the osteoplasty is for replacement of a joint selected from the group consisting of hip joint, knee joint, shoulder joint, and elbow joint.

20. The method of claim 16, further comprising incorporating leuco-crystal violet into the liquid acrylic bone cement, wherein the leuco-crystal violet provides visualization of the acrylic bone cement or the proton beam path during the osteoplasty procedure.

21. The method of claim 16, wherein the osteoplasty procedure is selected from the group consisting of kyphoplasty and vertebroplasty.

22. The method of claim 16, further comprising containing the liquid acrylic bone cement within an expandable plastic bladder prior to introduction into the bone defect site, wherein the plastic bladder prevents the liquid acrylic bone cement from spreading beyond an intended area.

23. The method of claim 16, wherein the bone defect site includes a pre-drilled hole in the bone, and wherein the method further comprises:
placing a screw in the bone; and
introducing the liquid acrylic bone cement to enhance bonding of the screw to the bone.

24. The method of claim 23, further comprising:
placing a plurality of screws in one or more bones;
aligning the plurality of screws; and
curing the liquid acrylic bone cement to secure the plurality of screws in their aligned positions.

25. The method of claim 16, wherein the method is for prophylactically strengthening normal bones.

26. A method for visualizing energy deposition from a proton beam in biological tissue during in vivo therapeutic treatment, comprising:
incorporating leuco-crystal violet dye into a biocompatible material placed within or adjacent to the biological tissue;
irradiating the biological tissue and the biocompatible material with a proton beam; and
observing a color change in the leuco-crystal violet dye in the biocompatible material, the color change indicative of energy deposition from the proton beam within the biological tissue and the biocompatible material.

27. The method of claim 26, wherein the biocompatible material is an acrylic resin.

28. A n agar containing substantially cylindrical plastic tube comprising,
said substantially cylindrical plastic tube is two-thirds filled with agar, forming an agar surface and with an airspace comprising one-third of the tube's volume,
wherein an interface between the agar surface and the airspace is planar and extends through the length of the tube parallel to the side of the substantially cylindrical plastic tube.
